(19) United States
(12) Patent Application Publication
(10) Pub. No.: US 2013/0210878 A1
(43) Pub. Date: Aug. 15, 2013

(54) BENDAMUSTINE COMPOSITIONS AND METHODS THEREFORE

(71) Applicant: Innopharma, Inc., (US)

(72) Inventors: Kumaresh Soppimath, Monmouth Junction, NJ (US); Udaya Toti, East Windsor, NJ (US); Satish Pejaver, Bridgewater, NJ (US); Navneet Puri, Bridgewater, NJ (US)

(73) Assignee: Innopharma, Inc., Piscataway, NJ (US)

(21) Appl. No.: 13/749,443

(22) Filed: Jan. 24, 2013

Related U.S. Application Data
(60) Provisional application No. 61/589,975, filed on Jan. 24, 2012.

(51) Int. Cl. A61K 47/02

(52) U.S. Cl. CPC A61K 47/02
USPC 514/394

(57) ABSTRACT

Aqueous Bendamustine formulations with improved stability are disclosed. Especially preferred formulations are low-dose ready-to-use liquid formulations in which Bendamustine is in a non-aqueous vehicle in combination with an aqueous phase that contains significant quantities of chloride.

This application claims priority to our copending U.S. provisional application with the Ser. No. 61/589975, which was filed Jan. 24, 2012, and which is incorporated by reference herein.

### FIELD OF THE INVENTION

The present invention relates to liquid ready-to-use pharmaceutical compositions that include Bendamustine, and especially to aqueous formulations with substantially improved stability of Bendamustine.

### BACKGROUND OF THE INVENTION

Bendamustine hydrochloride (1H-benzimidazole-2-butanoic acid, 5-[bis(2-chloro-ethyl)amino]-1 methyl-, monohydrochloride; C16H21Cl2N3O2.HCll structure below) is a well-known alkylating agent comprising a mechlorethamine group and a benzimidazole heterocyclic ring with a butyric acid substituent.

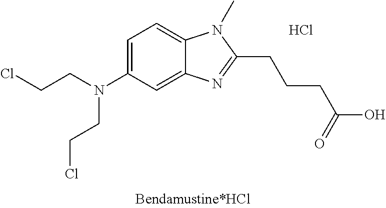 [SMILES: Cn1c(CCCC(=O)O)nc2cc(N(CCCl)CCCl)ccc21 ; Cl]

Bendamustine is thought to act as antimetabolic and cytotoxic agent and is typically prescribed for patients suffering from chronic lymphocytic leukemia (CLL) and indolent B-cell non-Hodgkin's lymphoma (NHL) that has progressed during or within six months of treatment with rituximab or a rituximab-containing regimen. Bendamustine hydrochloride is marketed in the US under the trademark TREANDA and is typically supplied as lyophilized powder that is reconstituted just prior to injection. Indeed, it is critical (as reflected in the prescribing information for TREANDA) that the reconstituted admixture should be prepared as close as possible to the time of patient administration as the final admixture is stable only for 24 hours when stored refrigerated (2-8° C. or 36-47° F.) or for 3 hours when stored at room temperature (15-30° C. or 59-86° F.) and room light. Administration of TREANDA must be completed within this period.

The reason for the significant lack of stability is thought to be rapid hydrolysis due to the presence of highly labile aliphatic chlorine atoms in Bendamustine and has been widely discussed in the literature. For example, US2012/0129904 teaches in [0023] that water and glycols rapidly degrade Bendamustine, and US20110184036A1 points out in [0005] that stability of Bendamustine is measured in hours when dissolved in water. Analytic protocols and quantification methods of Bendamustine and its hydrolysis products have been reported (e.g., J Chromatogr B Analyt Technol Biomed Life Sci. 2012 Apr. 15; 893-894:92-100).

Because of the patient-specific dosing, repeated administration, and a dosage regimen of six 28-day cycles, there is a strong need for ready-to-use formulations that will not only provide enhanced stability but also allow for a multi-dose formulation. Unfortunately, while various attempts have been made to stabilize Bendamustine, all or almost all of them suffer from several disadvantages. For example, certain non-aqueous solvent systems have been proposed in U.S. Pat. No. 8,344,006, U.S. 2012/0129904, U.S. 2011/0190363, U.S. 2012/0157505, and WO 2010/036702 to increase long-term stability. While at least some of these systems provide significant improvement, tolerability of the solvents by the patient is in at least some cases less than desirable. Moreover, the solution of most such systems is not ready-to-use and must be diluted prior to use to avoid possible adverse effects due to the solvents.

Similarly, as for example described in U.S. 2011/0184036 and WO 2011/094565, a highly concentrated Bendamustine solution was formulated with non-aqueous polyethylene glycol (PEG) and/or propylene glycol (PG) in combination with an antioxidant and/or chloride source. However, such solutions will again in most cases require significant dilution with an aqueous solvent prior to use, which tends to reduce ease of use. In still further known attempts, Bendamustine is combined with first charged cyclopolysaccharide and a stabilizing agent (which is a second charged cyclopolysaccharide) with a charge opposite to that of the first cyclopolysaccharide as discussed in WO 2012/127277, WO 2010/097700, and U.S. 2010/0216858. Similarly, U.S. 2011/0015245 teaches use of various amphiphilic cationic compositions to stabilize Bendamustine, while WO 2011/005714 teaches liposomal formulations encapsulating Bendamustine. Alternatively, Bendamustine is immobilized in a polymeric carrier to increase stability (J Pharm Biomed Anal. 2008 Dec. 1; 48(4):1143-50). However, such specialized formulations are often complex to manufacture and/or significantly increase the cost of production.

In yet another approach, stability of Bendamustine HCl (0.25 mg/ml in 0.9% sodium chloride) in water was studied. Here, Bendamustine was stable at 4° C. for 4 days and at 23° C. for 9 hours. While the temperature had adverse effects on stability, it was found that moderate concentrations of chloride ions increased the stability to at least some degree (see Pharmazie 1994; 49, 10: 775-777). However, such compositions were yet again not stable over extended periods, and thus provide at best limited use as a ready-to-use aqueous composition, even under refrigerated conditions.

Thus, even though numerous Bendamustine formulations are known in the art, there is still a need to provide improved formulations, especially those that allow for multi-dose and ready-to-use formulations for injection in which Bendamustine has increased stability.

### SUMMARY OF THE INVENTION

The present invention is directed to compositions and methods for ready-to-use liquid formulations in which Bendamustine is in an aqueous formulation that is formed from a non-aqueous vehicle in combination with an aqueous phase that contains significant quantities of chloride. Bendamustine has significantly improved stability in such aqueous formulations, even over extended periods.

In one aspect of the inventive subject matter, a ready-to-use liquid Bendamustine formulation for injection comprises a mixture of a non-aqueous solvent system and an aqueous chloride-containing water phase, wherein Bendamustine is present in the mixture in a therapeutically effective amount. Most preferably, the chloride in contemplated liquid Bendamustine formulations is present in the mixture at a precipitating amount with respect to Bendamustine in the water phase, and the non-aqueous solvent system is present in the mixture in an amount effective to prevent Bendamustine precipitation from the water phase. Most advantageously, the precipitating amount of chloride is also effective to stabilize Bendamustine as compared to a mixture without chloride.

It is further generally preferred that the non-aqueous solvent system comprises propylene glycol and/or polyethylene glycol, or essentially consists of propylene glycol or polyethylene glycol. While not limiting to the inventive subject matter, it is also preferred that the Bendamustine is present at a concentration of equal or less than 10 mg/ml, and more typically less than 5 mg/ml. In other aspects of the inventive subject matter, it is preferred that the Bendamustine concentration is at least 5 mg/ml, and more typically at least 10 mg/ml. With respect to suitable chloride concentrations it is preferred that the chloride is present at a concentration of at least 5 mg/ml, and more typically at least 14 mg/ml. Additionally, it is preferred that the aqueous chloride-containing water phase is present in an amount of at least 10 vol %, and more preferably at least 25 vol %.

Therefore, suitable formulations may comprise Bendamustine at a concentration of equal or less than 10 mg/ml, the aqueous chloride-containing water phase in an amount of at least 10 vol %, and may use as the non-aqueous solvent system propylene glycol, and may comprise chloride in the mixture at a concentration of at least 5 mg/ml.

Consequently, and viewed from a different perspective, the inventors also contemplate a reconstitution solution for lyophilized Bendamustine suitable to produce a ready-to-use liquid formulation for injection. Most preferably, the reconstitution solution will include a mixture of a non-aqueous solvent system (e.g., comprising propylene glycol or polyethylene glycol) and an aqueous chloride-containing water phase (e.g., at least 10 vol %), wherein the chloride is present in the mixture at a precipitating amount with respect to Bendamustine in the water phase, and wherein the non-aqueous solvent system is present in the mixture in an amount effective to prevent Bendamustine precipitation from the water phase. In especially preferred aspects, the precipitating amount of chloride (e.g., at least 5 mg/ml) is effective to stabilize Bendamustine in the mixture when the mixture is combined with the lyophilized Bendamustine.

With respect to increased stability it is typically preferred that the reconstitution solution is formulated to stabilize Bendamustine in the mixture such as to limit total degradation product formation after at least three days under refrigeration at between 2-8° C. to equal or less than 1.0%, more typically equal or less than 0.5%, and most preferably equal or less than 0.2% of total Bendamustine combined with the mixture.

Therefore, the inventors also contemplate a pharmaceutical kit for administration of Bendamustine that includes a quantity of lyophilized Bendamustine and contemplated reconstitution solutions. Most preferably, the quantity of lyophilized Bendamustine is sufficient for at least two (e.g., three, four, five, and even more) independent administrations. Likewise, the inventors contemplate multi-use ready-to-use liquid formulation for multiple independent injections as contemplated herein.

In another aspect of the inventive subject matter, the inventors contemplate a method of stabilizing Bendamustine in a ready-to-use liquid formulation for multiple independent injections in which in one step a mixture of a non-aqueous solvent system and an aqueous chloride-containing water phase is provided. Most preferably, the chloride is present in the mixture at a precipitating amount with respect to Bendamustine in the water phase, and the non-aqueous solvent system is present in the mixture in an amount effective to prevent Bendamustine precipitation from the water phase, wherein the precipitating amount of chloride is effective to stabilize Bendamustine in the mixture when the mixture is combined with the lyophilized Bendamustine. In another step, the Bendamustine is dissolved in a volume of the mixture to thereby form the ready-to-use liquid formulation for injection, wherein the volume is sufficient to form the ready-to-use liquid formulation for multiple independent injections.

Most preferably, Bendamustine in contemplated methods is present at a concentration of equal or less than 10 mg/ml, the aqueous chloride-containing water phase is present in an amount of at least 10 vol %, the non-aqueous solvent system comprises propylene glycol, and the chloride is present in the mixture at a concentration of at least 5 mg/ml. In contemplated methods, Bendamustine is therefore stabilized in the mixture to limit total degradation product formation after at least three days under refrigeration at between 2-8° C. to equal or less than 1.0% (and more typically equal or less than 0.5% and even less) of total Bendamustine combined with the mixture.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWING

FIG. 1 is a table showing exemplary stability data for Bendamustine in water.

FIG. 2 is a table showing exemplary stability data for Bendamustine in selected solvents and selected aqueous solvent mixtures with precipitating quantities of chloride.

FIG. 3 is a table showing exemplary stability data for Bendamustine in water using different chloride concentration, and selected aqueous solvent mixtures with precipitating quantities of chloride and different quantities of water.

FIG. 4 is a table showing exemplary stability data for Bendamustine in selected aqueous solvent mixtures with different precipitating quantities of chloride.

FIGS. 5A-5E are exemplary chromatographs of selected Bendamustine formulations.

### DETAILED DESCRIPTION

The inventors have unexpectedly discovered that aqueous Bendamustine formulations can be prepared in which Bendamustine has substantial stability, despite relatively high water content in the formulation. As is well known in the art, water severely compromises stability of Bendamustine by hydrolysis (Bendamustine typically degrades within hours). Moreover, while it was previously known that chloride ions could to a limited degree reduce hydrolysis of Bendamustine in water, protection against hydrolysis to achieve stable pharmaceutical compositions was considered as not feasible as increased quantities of chloride rapidly lead to precipitation of Bendamustine.

Remarkably, the inventors have now discovered that even relatively large quantities of water and otherwise precipitating quantities of chloride ions can be included in aqueous formulations where such formulations also include a co-solvent that solubilizes otherwise precipitating Bendamustine. Most advantageously, such co-solvent is completely miscible with water and pharmaceutically acceptable to so allow formation of a single-phase aqueous mixture in which Bendamustine remains largely protected, especially where the mixture is kept refrigerated between 2-8° C. Contemplated compositions and methods will also include additional ingredients, and especially preservatives, and antioxidants.

It is generally contemplated that the compositions according to the inventive subject matter will be liquid compositions that include a mixture of a non-aqueous solvent system and an aqueous chloride-containing water phase, which may further comprise Bendamustine in a therapeutically effective amount. It is also generally contemplated that the compositions presented herein are pharmaceutically acceptable and suitable for injection or (otherwise parenteral administration), and that the compositions are present in a single phase (i.e., will exhibit no phase separation into distinct phases upon storage at room temperature or under refrigeration), and most typically be free of solid or particulate matter (including liposomes).

With respect to suitable Bendamustine concentrations in the ready-to-use formulation, it is generally preferred that the Bendamustine concentration is relatively low, and in most cases at about 5 mg/ml. The term “about” when used in conjunction with a numeral here refers to a range of that numeral =/−10%, inclusive. However, alternative concentrations are also expressly deemed suitable for use herein. For example, suitable concentrations of Bendamustine in the ready-to-use formulation will be between 0.1 and 1 mg/ml, between 1.0 and 5 mg/ml, between 5 and 10 mg/ml, between 1 and 10 mg/ml, between 5 and 20 mg/ml, between 10 and 50 mg/ml, and between 20 and 70 mg/ml, and even higher. Lower concentrations are typically directly administered, while higher concentrations are added to intravenous saline solution. However, in especially preferred aspects, the Bendamustine concentration in the ready-to-use formulation is identical to the concentration as presently administered from reconstituted lyophilized preparations. Thus, particularly preferred concentrations of Bendamustine in the ready-to-use formulation are between 0.1 mg/ml and 5 mg/ml. It should of course be appreciated that the compositions and mixtures contemplated herein may also be employed as a reconstitution solution for lyophilized Bendamustine. Therefore, contemplated solutions expressly include those with or without Bendamustine.

There are numerous suitable solvents for the non-aqueous solvent system, however, especially preferred solvent systems will include a single non-aqueous solvent (i.e., water content less than 1 vol %), which is typically a polar, protic or non-protic solvent. Thus, especially contemplated solvents include various alcohols (and especially ethanol), various glycols (propylene glycol, polyethylene glycol, etc.), dimethylacetamide, N-methylpyroll-idone, and dimethylsulphoxide. Where desired, the non-aqueous solvent system may also be prepared from two or more solvents, which are again most preferably polar, protic or non-protic solvents as noted above. Of course, it should be appreciated that pharmaceutically acceptable solvents are especially preferred for use herein.

Most preferably, the non-aqueous solvent system is present in an amount that is sufficient to solubilize all of the Bendamustine that would otherwise precipitate from the aqueous phase at the particular chloride (or other anion) concentration, and the person of ordinary skill will be readily appraised of suitable quantities for the non-aqueous solvent system without undue experimentation. Therefore, the ratio of the non-aqueous solvent system to the aqueous chloride-containing water phase may vary considerably. However, the non-aqueous solvent system will typically equal or more than 20 vol %, more typically more equal or than 30 vol %, even more typically equal or more than 40 vol %, and most typically equal or more than 50 vol %.

With respect to the aqueous chloride-containing water phase it is contemplated that the aqueous chloride-containing water phase will preferably include only water and the chloride (plus the accompanying cation), but may also include water-miscible co-solvents next to the water. Examples of suitable co-solvents include tert-butyl alcohol, methanol, ethanol. Most preferably, where the aqueous chloride-containing water phase includes more than water, the water phase will be a homogenous mixture that does not form one or more phase boundaries. It is further generally preferred that the aqueous chloride-containing water phase is present in a relatively high amount, and most preferably in the maximum amount suitable for a desired stability. Therefore, the aqueous chloride-containing water phase will be present in the ready-to-use liquid in an amount of at least 10 vol %, more typically at least 20 vol %, even more typically at least 35 vol %, and most typically at least 50 vol %.

It is further generally preferred that the chloride species may be added via any salt and suitable salts include hydrochloric acid and any inorganic salt thereof (e.g., NaCl, BaCl, KCl, MgCl₂, etc) and/or organic salts having a chloride anion (e.g., choline chloride, ammonium chloride, etc.). It should be further appreciated that various alternative anionic species may be used and suitable alternate anionic species include various halides (e.g., bromide, iodide, etc.), but also organic anionic species such as nitrates, carbonates, acetates, mesylates, etc., and all reasonable mixtures thereof.

Particularly preferred concentrations of the chloride (and/or other anionic species) are precipitating concentrations with respect to Bendamustine in water only. Thus, viewed from another perspective, suitable chloride concentrations will be those needed to exceed the solubility limit for a given Bendamustine concentration in water at room temperature and a pH of about pH 3. Consequently, and most typically, preferred concentrations of the chloride will be at least 5 mg/ml, more typically at least at least 14 mg/ml, and most typically at least 29 mg/ml, particularly where the Bendamustine concentration is equal or less than 100 mg/ml, and more typically equal or less than 50 mg/ml, even more typically equal or less than 10 mg/ml, and most typically equal or less than 5 mg/ml. Viewed from a different perspective, it is generally preferred that NaCl (or other chloride or anionic species) is present in molar excess relative to Bendamustine Hydrochloride. For example, the molar ratio of Bendamustine Hydrochloride to NaCl is preferably at least 1:2, more preferably at least 1:3, even more preferably at least 1:5, still more preferably at least 1:7, and most preferably at least 1:15 and above.

In still further contemplated aspects, additional ingredients are used in contemplated compositions at appropriate concentrations, and particularly preferred additional ingredients include preservatives, antioxidants, isotonicity agents, and all reasonable mixtures thereof. For example, suitable antioxidants include hydrophobic anti-oxidants (e.g., butylated hydroxytoluene, butylated hydroxyanisole, propyl gallate, and α-tocopherol, α-tocopherol Tocopherol Polyethylene Glycol Succinate (Vitamin E TPGS), L-cysteine), or water soluble anti-oxidants (e.g., sodium EDTA and thioglycerol). Most typically, preferred anti-oxidant concentrations will be between 0.005 wt % and 5 wt % of the total composition. Likewise, tonicity may be adjusted (preferably to isotonicity) as is well known in the art, and all known preservatives at their respective known concentrations are deemed suitable.

While the pH of contemplated formulations may vary widely and be between 1 and 14, it is preferred that the pH is suitable for parenteral administration, and especially suitable for injection. Therefore, preferred pH values will be in the range of 3-10, and more typically between 4.5 and 8.5. Most typically, where pH control is desired, one or more buffers may be used in the ready-to-use formulation, and suitable buffers include all pharmaceutically acceptable buffers. For example, preferred buffers include citrate buffers, acetate buffers, maleate buffers, phosphate buffers, succinate/tartrate buffers, typically having a buffer strength of between 5 mM and 150 mM. It is still further preferred that the compositions according to the inventive subject matter are sterile. To that end, the compositions may be filtered through a 0.22 micron filter, and/or be subjected to heat, radiation (e.g., gamma, electron beam, microwave), ethylene oxide sterilization at suitable dosages to achieve a desired sterility level.

Most typically, the inventors discovered that when they dissolved Bendamustine in contemplated compositions, an increase in stability is found at all temperatures, including under refrigeration (2-8° C.), ambient/room temperature (18-22° C.), accelerated conditions (40° C.), and super accelerated conditions (50° C.), especially where stored in an amber (or otherwise light-protected) glass vial under inert gas headspace. The increase in stability is in most cases evident over extended periods, and can typically be seen over a period of at least three days, at least seven days, at least two weeks, at least one month, at least six months, and at least a year and even longer. For example, contemplated stability can be evidenced as having a total degradation product of equal or less than 10%, more typically equal or less than 5%, even more typically equal or less than 3%, and most typically equal or less than 1-2% over an extended period of time (e.g., at least three days, at least seven days, at least two weeks, at least one month, or at least three months under refrigeration or room temperature).

Stability is generally assessed by chromatographic analysis and is in most cases expressed as presence/quantity of total degradation products relative to quantity at start of dissolution of Bendamustine. Most typically, quantity of total degradation products can be calculated by AUC (area under the curve) of the chromatography. Alternatively, stability may also be expressed as % retention of undegraded Bendamustine after predetermined time course. In still further alternative measures, the stability can be expressed as quantity of known hydrolysis products (e.g., monohydroxylated form and/or dihydroxylated form), alone or as a ratio with undegraded Bendamustine. As already noted before, stability is typically determined upon termination of certain storage conditions, including refrigerated (2-8° C.), ambient/room temperature (18-22° C.), accelerated conditions (40° C.), and super accelerated conditions (50° C.), preferably in an amber (or otherwise light-protected) glass vial under inert gas headspace. Based on the inventors' observations and further experimental data (e.g., see below), Bendamustine is stabilized in the mixture to limit total degradation product formation after at least three days, more preferably after at least seven days, and most preferably after at least two weeks under refrigeration at between 2-8° C. to equal or less than 1.0%, more preferably equal or less than 0.5%, and most preferably equal or less than 0.2% of total Bendamustine combined with the mixture.

Therefore, the inventors contemplate kits with or without Bendamustine that provide the reconstitution solution and optionally lyophilized Bendamustine to a care giver or other medical professional, wherein the Bendamustine has increased storage stability in aqueous medium. As contemplated compositions and methods are particularly suitable for multiple independent administrations of Bendamustine, suitable quantities of the reconstitution solution and Bendamustine are contemplated, which will typically be sufficient for at least two, more typically at least five, and most typically at least ten independent administrations. Thus, it should be appreciated that Bendamustine can be formulated or reconstituted from a lyophilized to form an aqueous, ready-to-use formulation in which Bendamustine is stable over an extended period to allow storage and multi-use of the ready-to-use formulation over at least two, at least five, and most preferably at least ten independent administrations from the same ready-to-use formulation. The term “ready-to-use formulation” or “ready-to-use liquid” are used interchangeably herein and mean that the formulation or liquid is suitable for therapeutic administration without further dilution or combination with another ingredient prior to administration.

Viewed from another perspective, it should be recognized that Bendamustine can be stabilized (and particularly stabilized in a ready-to-use aqueous liquid formulation for multiple independent injections) by providing a mixture of a non-aqueous solvent system and an aqueous chloride-containing water phase, wherein the chloride is present in the mixture at a precipitating amount with respect to Bendamustine in the water phase, and wherein the non-aqueous solvent system is present in the mixture in an amount effective to prevent Bendamustine precipitation from the water phase, and wherein the precipitating amount of chloride is effective to stabilize Bendamustine in the mixture when the mixture is combined with the lyophilized Bendamustine. Bendamustine is then dissolved in a volume of the mixture to thereby form the ready-to-use liquid formulation for injection, wherein the volume is sufficient to form the ready-to-use liquid formulation for multiple independent injections.

### EXAMPLES

The following is provided to illustrate various exemplary aspects of the inventive subject matter. More specifically, various Bendamustine preparations are presented that differ in the water content, chloride content, and stability of the respective preparations.

Analytical Protocol: An analytical method for Bendamustine Hydrochloride assay and related substances was developed at Innopharma using a HPLC gradient elution method. More specifically: Column used was Waters Atlantis dC184.6 mm×150 mm, 3 μm with a column Temperature of 30° C. and a sample Temperature of 5° C. Run time was 50 minutes with integration time of 42 minutes. Detection Wavelength was 245 nm at a flow rate of 1.0 mL/min Injection volume was 10 μL and needle wash was performed with methanol. Column wash was performed with 70:30 acetonitrile:water. Mobile phase A was 0.05% (v/v) TFA in water, and Mobile phase B was 0.05% (v/v) TFA in acetonitrile. Gradient composition and time was as noted in Table 0 below (Diluent was methanol, and blank solution was methanol).

TABLE 0

| Time | % A | % B | Curve |
|---|---|---|---|
| 0 | 90.0 | 10.0 | — |
| 10.0 | 90.0 | 10.0 | (Isocratic) |
| 40.0 | 50.0 | 50.0 | (Linear) |
| 45.0 | 90.0 | 10.0 | (Immediate change) |
| 50.0 | 90.0 | 10.0 | (Isocratic) |

Sample Preparation was performed as follows: Pipette 2.0 mL of the bulk solution (approximately 5 mg/mL active) into a 50-mL tared volumetric flask. Accurately weigh and record the weight of the bulk solution. Add methanol to volume and mix well. The sample solution contains a nominal concentration of 0.2 mg/mL Bendamustine HCl. Inject the solution onto HPLC.

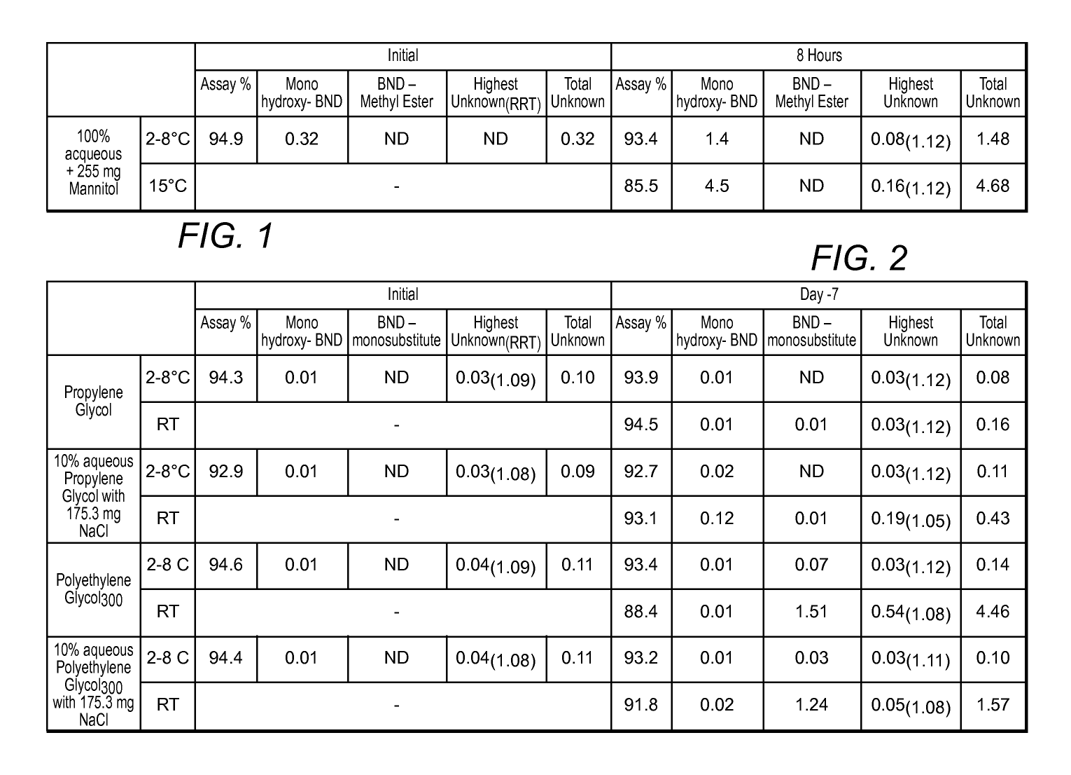

Example 1: Formulation 0 was prepared by dissolving Bendamustine hydrochloride in water suitable for injection containing Mannitol to make a total of 30 mL ready to use single dose injection in vial as shown in Table 1. Typical stability results for Formulation 0 are illustrated in the Table of FIG. 1. As can be readily seen from the results, Bendamustine degrades to a significant degree within hours under both storage conditions, refrigeration and near room temperature storage. Clearly, such formulation is not suitable for multi-use over several days or weeks.

TABLE 1

| Ingredients | Formulation 0 | |
|---|---|---|
| Bendamustine HCl | 150 | mg |
| Mannitol | 255 | mg |
| DI Water | 30 | mL |

Example 2: In several experiments replacement of water with an organic solvent was performed, with the optional addition of NaCl at very high concentrations. Table 2 shows the exemplary composition of such formulations. More specifically, Formulation I was prepared by dissolving Bendamustine hydrochloride in 30 mL polyethylene glycol 300 at 0-3° C. to make 30 mL ready to use single dose injection in a 30 mL amber vial; Formulation II was prepared by dissolving Bendamustine hydrochloride in 30 mL propylene glycol at 0-3° C. to make 30 mL ready to use single dose injection in a 30 mL amber vial; Formulation III was prepared by dissolving Bendamustine hydrochloride in 27 mL polyethylene glycol 300 at 0-3° C. and add 3 mL aqueous phase containing 175.3 mg NaCl to make 30 mL ready to use single dose injection in a 30 mL amber vial; and Formulation IV was prepared by dissolving Bendamustine hydrochloride in 27 mL propylene glycol at 0-3° C. and add 3 mL aqueous phase containing 175.3 mg NaCl to make 30 mL ready to use single dose injection in a 30 mL amber vial.

TABLE 2

| | Formu- | | Formu- | | Formu- | | Formu- | |
|---|---|---|---|---|---|---|---|---|
| Ingredients | lation I | | lation II | | lation III | | lation IV | |
| Bendamustine HCl | 150 | mg | 150 | mg | 150 | mg | 150 | mg |

| | | | | | | |
|---|---|---|---|---|---|---|
| Propylene Glycol (PG) | — | 30.0 | mL | — | 27.0 | mL |

| | | | | | |
|---|---|---|---|---|---|
| Polyethylene glycol 300 | 30.0 | mL | 27.0 | mL | — |

| | | | | | | |
|---|---|---|---|---|---|---|
| Sodium Chloride | — | — | 175.3 | mg | 175.3 | mg |
| DI Water | — | — | 3.0 | mL | 3.0 | mL |

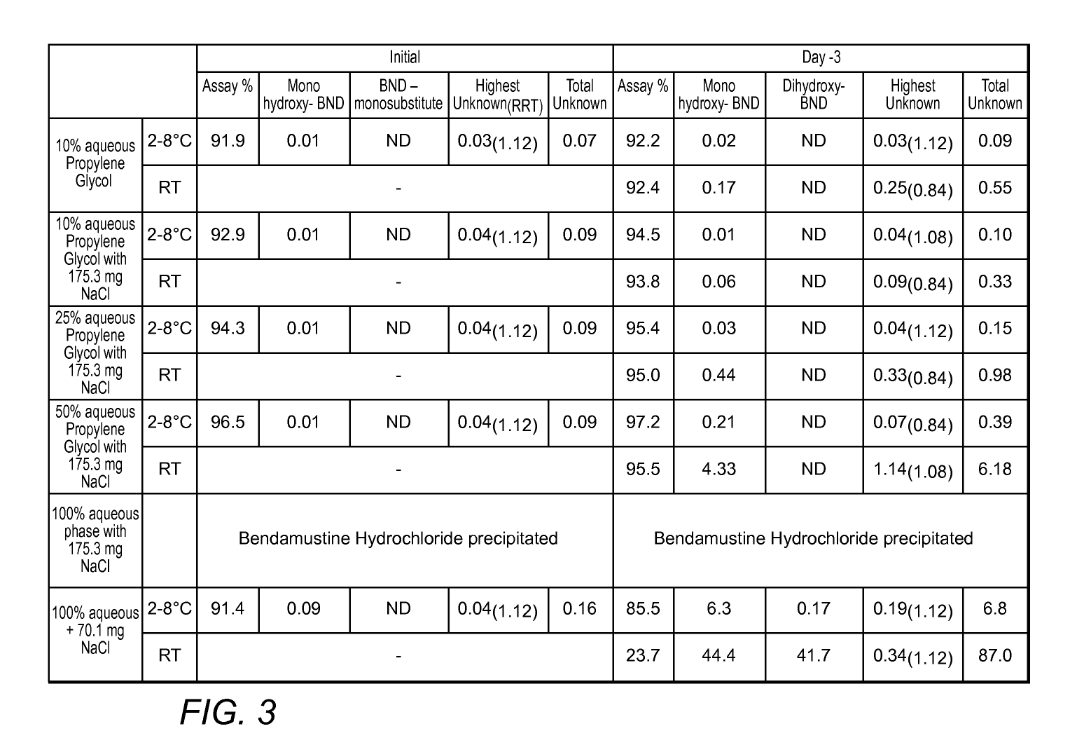

The stability study results are presented in the Table of FIG. 2. Remarkably, Bendamustine was significantly stabilized by replacement of water with a glycol solvent, despite the nucleophilic character of the glycol reagent. Even more remarkable was the fact that Bendamustine could be stabilized at relatively high percentage of water in the mixture that would otherwise have precipitated the Bendamustine out of solution as can be seen in more detail below.

Example 3: To investigate the role of water and chloride, further formulations were prepared as shown in Tables 3 and 4. Here, Bendamustine hydrochloride for Formulations V to VII was dissolved in propylene glycol and the aqueous phase was added with and without sodium chloride to make 30 mL ready to use single dose injection in a 30 mL amber vial. Formulation VIII was prepared by dissolving Bendamustine hydrochloride in 29 mL DI water and add 1.2 mL aqueous composition containing 70.1 mg of sodium chloride to make 30.2 mL ready to use single dose injection in a 30 mL amber vial; Formulation IX was prepared by dissolving Bendamustine hydrochloride in 27 mL DI water and add 3 mL aqueous composition containing 175.3 mg of sodium chloride to make 30.0 mL ready to use single dose injection in a 30 mL amber vial.

TABLE 3

| Ingredients | Formulation V | | Formulation VI | | Formulation VII | |
|---|---|---|---|---|---|---|
| Bendamustine HCl | 150 | mg | 150 | mg | 150 | mg |
| Sodium Chloride | — | | 175.3 | mg | 175.3 | mg |
| DI Water | 3.0 | mL | 7.5 | mL | 15.0 | mL |
| Propylene glycol | 27.0 | mL | 22.5 | mL | 15.0 | mL |
| (PG) | | | | | | |

TABLE 4

| | | |
|---|---|---|
| Ingredients | Formulation VIII | Formulation IX |

| | | | | |
|---|---|---|---|---|
| Bendamustine HCl | 150 | mg | 150 | mg |
| DI water | 30.0 | mL | 30.2 | mL |
| Sodium Chloride | 175.3 | mg | 70.1 | mg |

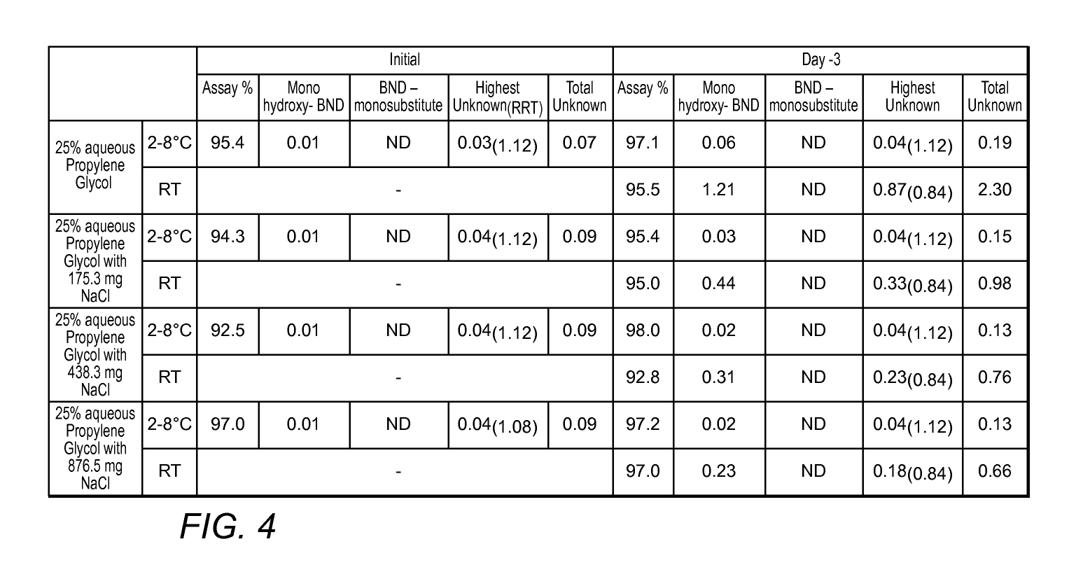

Interestingly, and as shown in the table of FIG. 3, even substantial increases of the water in the mixture (here: up to 50 vol %) did not substantially increase the total degradation as one would have expected. Of further particular relevance is that in water non-precipitating quantities of chloride did not exhibit desirable protective effect against degradation. However, at otherwise precipitating quantities of chloride, substantial protective effect was observed in aqueous solvent mixtures. While not wishing to be bound by any theory or hypothesis, the inventors contemplate that the addition of a co-solvent provided sufficient solubilizing power to the mixed phase system such that otherwise precipitating quantities of Bendamustine was stabilized and so could receive a protective effect that would not have been otherwise available due to solubility limitations.

Example 4: Further variations to increase chloride concentration in the solvent system are exemplarily illustrated in Table 5 below. Here, Bendamustine hydrochloride was dissolved in 22.5 ml Propylene glycol and add 7.5 mL aqueous phase to make 30 mL ready to use single dose injection in a 30 mL amber vial .

TABLE 5

| Ingredients | Formulation X | | Formulation XI | | Formulation XII | |
|---|---|---|---|---|---|---|
| Bendamustine HCl | 150 | mg | 150 | mg | 150 | mg |
| Sodium Chloride | — | | 438.3 | mg | 876.5 | mg |
| Propylene glycol | 22.5 | mL | 22.5 | mL | 22.5 | mL |
| DI Water | 7.5 | mL | 7.5 | mL | 7.5 | mL |

Table 6 depicts exemplary molar ratios of Bendamustine to NaCl and Cl— to NaCl in selected above formulations:

TABLE 6

| | Molar ratio of Bendamustine | Molar ratio of |
|---|---|---|
| NaCl Amount | Hydrochloride to NaCl | Cl⁻ to NaCl |
| 70.1 mg | 1:3.16 | 1:1.05 |
| 175.3 mg | 1:7.9 | 1:2.6 |
| 438.3 mg | 1:19.7 | 1:6.6 |
| 876.5 mg | 1:39.5 | 1:13.2 |

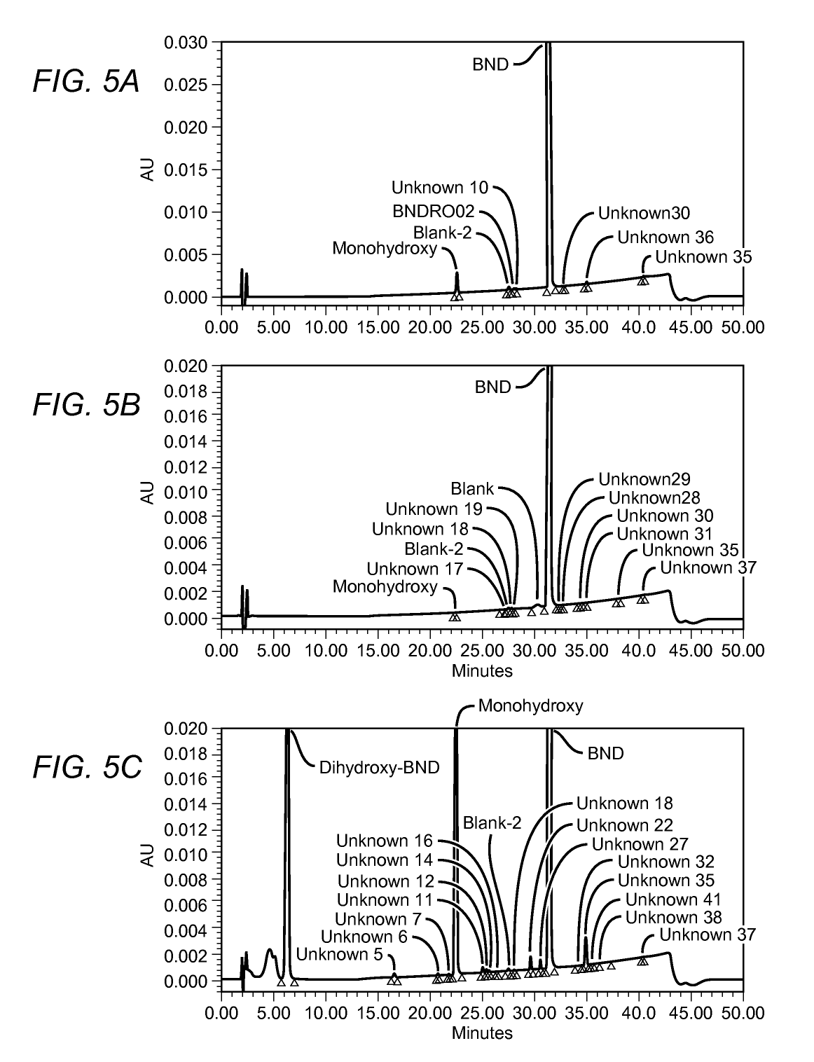

Notably, as is evident from the table in FIG. 4, protection of Bendamustine was increased by even higher chloride concentrations in the presence of significant quantities of water. In this case, increased protection was even apparent under room temperature storage conditions. Consequently, it should be appreciated that aqueous Bendamustine formulations with significantly improved stability can be prepared that have surprisingly high stability in even highly aqueous environment using chloride concentrations that would otherwise precipitate Bendamustine and so negate beneficial effects of chloride.

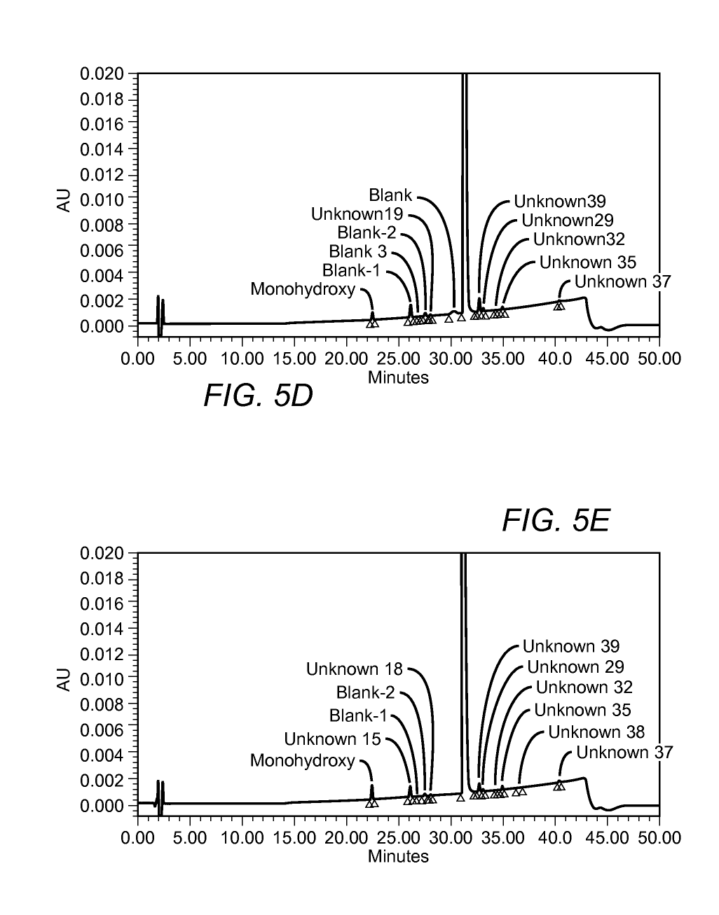

FIGS. 5A-5E depict exemplary chromatograms of selected compositions. More specifically: FIG. 5A is a chromatogram for Formulation 0 at initial conditions; FIG. 5B is a chromatogram for Formulation II at initial conditions; FIG. 5C is a chromatogram for Formulation IX at day 3 when stored at room temperature; FIG. 5D is a chromatogram for Formulation IV at day 7 when stored at room temperature; and FIG. 5E is a chromatogram for Formulation XII at day 3 when stored at room temperature.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A ready-to-use liquid Bendamustine formulation for injection, comprising:
a mixture of a non-aqueous solvent system and an aqueous chloride-containing water phase;
wherein Bendamustine is present in the mixture in a therapeutically effective amount;
wherein the chloride is present in the mixture at a precipitating amount with respect to Bendamustine in the water phase, and wherein the non-aqueous solvent system is present in the mixture in an amount effective to prevent Bendamustine precipitation from the water phase; and
wherein the precipitating amount of chloride is effective to stabilize Bendamustine as compared to a mixture without chloride.

2. The formulation of claim 1 wherein the non-aqueous solvent system comprises at least one of propylene glycol or polyethylene glycol.

3. The formulation of claim 1 wherein the non-aqueous solvent system essentially consists of propylene glycol or polyethylene glycol.

4. The formulation of claim 1 wherein the Bendamustine is present at a concentration of at least 5 mg/ml.

5. The formulation of claim 1 wherein the Bendamustine is present at a concentration of equal or less than 5 mg/ml.

6. The formulation of claim 1 wherein the chloride is present at a concentration of at least 5 mg/ml.

7. The formulation of claim 1 wherein the chloride is present at a concentration of at least 14 mg/ml.

8. The formulation of claim 1 wherein the aqueous chloride-containing water phase is present in an amount of at least 10 vol %.

9. The formulation of claim 1 wherein the Bendamustine is present at a concentration of equal or less than 5 mg/ml, wherein the aqueous chloride-containing water phase is present in an amount of at least 10 vol %, wherein the non-aqueous solvent system comprises propylene glycol, and wherein the chloride is present in the mixture at a concentration of at least 5 mg/ml.

10. A reconstitution solution for lyophilized Bendamustine to thereby produce a ready-to-use liquid formulation for injection, comprising:
a mixture of a non-aqueous solvent system and an aqueous chloride-containing water phase;
wherein the chloride is present in the mixture at a precipitating amount with respect to Bendamustine in the water phase, and wherein the non-aqueous solvent system is present in the mixture in an amount effective to prevent Bendamustine precipitation from the water phase; and
wherein the precipitating amount of chloride is effective to stabilize Bendamustine in the mixture when the mixture is combined with the lyophilized Bendamustine.

11. The reconstitution solution of claim 10, wherein the precipitating amount of chloride is at least 5 mg/ml.

12. The reconstitution solution of claim 11, wherein the non-aqueous solvent system comprises at least one of propylene glycol or polyethylene glycol and wherein the aqueous chloride-containing water phase is present in an amount of at least 10 vol %.

13. The reconstitution solution of claim 12, wherein the Bendamustine is stabilized in the mixture to limit total degradation product formation after at least three days under refrigeration at between 2-8° C. to equal or less than 1.0% of total Bendamustine combined with the mixture.

14. The reconstitution solution of claim 12, wherein the Bendamustine is stabilized in the mixture to limit total degradation product formation after at least three days under refrigeration at between 2-8° C. to equal or less than 0.5% of total Bendamustine combined with the mixture.

15. The reconstitution solution of claim 12, wherein the Bendamustine is stabilized in the mixture to limit total degradation product formation after at least three days under refrigeration at between 2-8° C. to equal or less than 0.2% of total Bendamustine combined with the mixture.

16. A pharmaceutical kit for administration of Bendamustine, comprising a quantity of lyophilized Bendamustine and the reconstitution solution of claim 10.

17. The pharmaceutical kit of claim 16, wherein the quantity of lyophilized Bendamustine is sufficient for at least two independent administrations.

18. A method of stabilizing Bendamustine in a ready-to-use aqueous liquid formulation for multiple independent injections, comprising:
providing a mixture of a non-aqueous solvent system and an aqueous chloride-containing water phase;
wherein the chloride is present in the mixture at a precipitating amount with respect to Bendamustine in the water phase, and wherein the non-aqueous solvent system is present in the mixture in an amount effective to prevent Bendamustine precipitation from the water phase;
wherein the precipitating amount of chloride is effective to stabilize Bendamustine in the mixture when the mixture is combined with the lyophilized Bendamustine; and
dissolving the Bendamustine in a volume of the mixture to thereby form the ready-to-use liquid formulation for injection, wherein the volume is sufficient to form the ready-to-use liquid formulation for multiple independent injections.

19. The method of claim 1, wherein the Bendamustine is present at a concentration of at least 5 mg/ml, wherein the aqueous chloride-containing water phase is present in an amount of at least 10 vol %, wherein the non-aqueous solvent system comprises propylene glycol, and wherein the chloride is present in the mixture at a concentration of at least 5 mg/ml.

20. The method of claim 17, wherein the Bendamustine is stabilized in the mixture to limit total degradation product formation after at least three days under refrigeration at between 2-8° C. to equal or less than 1.0% of total Bendamustine combined with the mixture.
